(19) United States
(12) Patent Application Publication
(10) Pub. No.: US 2021/0062124 A1
(43) Pub. Date: Mar. 4, 2021

(54) PHOTOBIOREACTOR FOR CONTAINED MICROORGANISM CULTIVATION

(71) Applicant: Synthetic Genomics, Inc., La Jolla, CA (US)

(72) Inventors: Miguel Olaizola, La Jolla, CA (US); Jason Davenport, La Jolla, CA (US); Julie Coard, La Jolla, CA (US); Xavier Perez, La Jolla, CA (US); Louis R. Brown, La Jolla, CA (US); Robert C. Brown, La Jolla, CA (US)

(21) Appl. No.: 16/960,789

(22) PCT Filed: Dec. 4, 2018
(86) PCT No.: PCT/US18/63920

§ 371 Date: Jul. 8, 2020

Related U.S. Application Data
(60) Provisional application No. 62/594,456, filed on Dec. 4, 2017.

(51) Int. Cl. C12M 1/00; C12M 1/12

(52) U.S. Cl. CPC C12M 21/02; C12M 23/06; C12M 29/06; C12M 29/20; C12M 23/26

(57) ABSTRACT

At least one elongated photobioreactor, at a small angle relative to horizontal and mixed substantially or entirely by large bubble flow, is used for contained cell culture, e.g., microalgae cultivation. Elongated, flexible, transparent, polymeric photobioreactor tubes, in near-grade and near-horizontal (e.g. sloped 1 degree to 3 degrees) orientation, and use of low-pressure air mixing, allow very inexpensive construction and operation. Multiple elongated tubes may be used for an independent operation of the multiple photobioreactor tubes for the same or different cells, e.g., microalgae and different applications. Low-pressure air is delivered near the low end of the bioreactor at less than 10 psig and without sparging, to produce large air bubbles that travel from the low end to the high end of the bioreactor, for turbulent mixing and gas exchange. Each inexpensive, flexible bioreactor tube is easily modified to improve internal flow characteristics and suspension of cells, and/or to include sensor and/or sampling collars and ports.

This application claims priority of Provisional Application Ser. No. 62/594456, filed Dec. 4, 2017 and entitled “Photobioreactor for Contained Microorganism Cultivation”, the entire disclosure of which, including the Appendices to the Specification, is incorporated herein by this reference.

### FIELD OF THE TECHNOLOGY

The technology relates to photobioreactors (PBR) for cultivation of microorganisms. More specifically, the technology provides an economically-constructed and economically-operated, controlled and enclosed growth environment, for the production of microorganisms, e.g., microalgal feedstocks, cyanobacteria, chytrid in a variety of applications for a variety of commercial products.

### SUMMARY OF THE TECHNOLOGY

The photobioreactor system comprises at least one elongated bioreactor, for containing a suspension of microorganisms in water/growth-media. The elongated bioreactor extends at a small angle relative to horizontal, so that it is sloped but close to horizontal. In certain embodiments, the bioreactor relies on low-pressure air flowing longitudinally through the bioreactor main body for mixing and oxygen stripping. For example, the elongated bioreactor may slope at one or more angles in the range of about 1-8 degrees, about 1-5 degrees, or about 1-3 degrees. For example, low-pressure air may be delivered at or near the low end of the tubular bioreactor at less than 10 psig, so that the delivered air pressure is slightly above the liquid head pressure in the bioreactor. The air flow and air-inlet are adapted to produce large air bubbles in the lower end of the bioreactor that travel from the low end to the high end of the bioreactor. Mixing occurs along the length of the bioreactor due to the longitudinal movement of a series of these large bubbles through the bioreactor interior space from the head piece to the tail piece of the bioreactor.

In certain embodiments, the bioreactor main body may be described as a tube, wherein large bubbles flow in quick succession into the tube and along the length of the tube to the high end of the tube. The large bubbles tend to flow along the interior longitudinal top surface of the tube to fill a substantial portion of the tube interior space near that longitudinal top surface. The high frequency of bubble production and the large size of the bubbles result in turbulent mixing that provides good mass transfer and cell suspension, and prevents settling of cells from the cell suspension, while causing little cell damage. Thus, this large-bubble mixing benefits cell growth by improving cell access to light, thereby enhancing productivity as well as providing access to nutrients and CO2 and by improving stripping of oxygen.

Certain embodiments are adapted to not divide large bubbles into small bubbles in the air-inlet or the low-end “head piece” of the bioreactor tube. For example, certain embodiments omit devices from the air-inlet and head piece that would break up the air volume into small bubbles, for example, omitting small-bubble-creating devices such as sparger plates, nozzles, orifice plates, baffles, and protrusions into the air-inlet and head piece. This way, a large volume of air gathers in the head piece, and, when that air pressure overcomes the hydrostatic head at the low end of the bioreactor tube, one large bubble at a time will “pop” or “burp” or “blurp” from the head piece into the main body of the bioreactor tube and travel through the bulk of the suspension, to the high end or “tail” of the tube. Along the way, each large bubble mixes the culture/suspension, creating turbulent fluid zones around each bubble.

In addition to, or instead of, the air-inlet and head piece not including small-bubble-creating devices, certain embodiments are adapted to not divide large bubbles into small bubbles after the large bubbles exit the air-inlet and the head piece. For example, certain embodiments omit small-bubble-creating devices from the bioreactor tube between the head piece and the tail piece, for example, omitting devices such as sparger plates, nozzles, orifice plates, baffles, and protrusions that break up the large bubbles that form in the head piece. Certain other embodiments omit devices for small-bubble-creating devices from the bioreactor tube between the head piece and the tail piece, for example omitting devices such as sparger plates, nozzles, and baffles, but include one or more sensing devices, sampling probes, and/or fluid-exit ports; for example, one or more sensor probes and/or sampling probes may be inserted permanently or temporarily to measure/monitor temperature, pH, oxygen content, etc., or to withdraw a sample of the bioreactor fluid for lab analysis. Additionally or instead, certain embodiments omit small-bubble-creating devices from the tail piece, for example omitting devices such as sparger plates, nozzles, orifice plates, baffles, and protrusions, but include a gas-exit port for bioreactor gasses to flow from the tail piece to a bleach trap or other neutralizing/scrubbing system.

Certain embodiments comprise adaptations in the tubular bioreactor system for further increasing effectiveness of the large-bubble mixing. In certain embodiments, these adaptations may adjust/control the path and/or location of the large bubbles, forcing the large bubbles to travel closer to, and/or along, the interior longitudinal bottom surface of the bioreactor tube in one or more locations along the tube length. In certain embodiments, these adaptations may elevate, tilt, or otherwise move the bioreactor tube to urge settled cells into suspension. For example, these adaptations may include baffles inside the interior of the bioreactor tube; members/force on the exterior of the flexible bioreactor tube that shape/contour the tube wall/inner surface to serve as baffled/constricted areas; areas of reduced or otherwise-modified tube diameter or shape, spaced-apart along the tube length; mechanical force exerted on one or more portions of the bioreactor tube; and/or mechanical movement of the bioreactor tube.

Multiple bioreactor tubes may be operated substantially or entirely separately and independently. Each bioreactor tube may be a system with no liquid flow from one bioreactor tube to any other bioreactor tube. For example, it is preferred that there is no fluid communication between multiple bioreactor tubes, except perhaps for air and CO2, from a single air source and a single CO2 source, respectively, being provided to multiple bioreactor tubes by means of separate control, separate one-way valving, and separate filtration, to prevent contamination between bioreactor tubes via the air or CO2 lines. Therefore, in certain embodiments, multiple bioreactor tubes are provided in a multi-tube system, wherein the multiple bioreactor tubes are side-by-side and parallel to each other but configured so that the cell suspension inside each bioreactor tube cannot flow to or reach any other of the bioreactor tubes. Therefore, in such embodiments, flow of air, cell suspension, and any other fluid inside a given tube may be described is in a single direction, from the head end to the tail end of that tube, and not to any adjacent tube.

In certain embodiments, cultivation of cells in each tubular bioreactor is a batch operation, or a semi-continuous operation where a portion of the cell suspension is periodically withdrawn/drained from the tubular bioreactor, for example through fill/harvest line F, for a partial harvest while the remaining cells continue to grow. In certain embodiments, there is no continuous flow of cell suspension into or out of the tubular bioreactor, and no flow, or no significant flow, of liquid inside the tubular reactor except for the large-bubble mixing discussed herein. In certain embodiments, gas flow into, through, and out from each individual bioreactor tube is continuous or semi-continuous, because air and CO2 are added continuously or semi-continuously to the interior of the tubular bioreactor. —Those gases, plus oxygen produced by the cells, flow to and typically continuously exit from the high end of said each individual bioreactor tube to a bleach trap or other neutralizing/scrubbing system provided for, and connected to, only that bioreactor tube.

Flexible translucent tubes, for example, thin-walled polyethylene or other polymeric tubes, have been round to be especially beneficial in serving as the main body of each bioreactor. Translucent photobioreactor tubes are beneficial for cell growth, and, in certain embodiments, the tubes are not only translucent but also transparent so that light enters the tubes and a viewer may see into the tubes to view/monitor the cell suspension. Thin-walled polymeric tubes may be supported by conventional and inexpensive support structures, and have been found to be particularly inexpensive in terms of initial equipment cost, handling cost, and installation cost. Further, such flexible tubes may be especially-well adapted for improved large-bubble mixing, as mentioned above in this document, by implementation of certain methods and apparatus that adjust/control the path and/or location of the large bubbles, force the large bubbles to travel along the interior longitudinal bottom surface of the tube, and/or elevate or otherwise move the tube to move settled cells into suspension.

These and other features, operation steps, and/or objects of the photobioreactor for contained microorganism cultivation are described below or will be understood by those of skill in this field from this document and the provisional application including appendices incorporated herein.

### BRIEF DESCRIPTION OF THE DRAWINGS

FIGS. 1-25 include representations, including schematic representations, of certain embodiments of apparatus and/or methods of the disclosed photobioreactor (PBR) system for contained microorganism cultivation. Microorganisms/cells are shown schematically, and given reference number 48′, only in FIG. 3, but it will be understood that the microorganisms/cells are in suspension in the cultivation culture inside the tube/tube portions 20 in other of the figures, and, in some circumstances/embodiments, are settled to the interior longitudinal bottom/bottom surface 26 of the tube/tube portions 20.

FIG. 1 is a side (elevation) view of one embodiment of the PBR system, operating with large-bubble mixing in the bioreactor tube.

FIG. 2 is a detail view of the area circled of FIG. 1.

FIG. 3 is a transverse (radial) cross-section of the embodiment of FIG. 1, viewed along the line 3-3 in FIG. 1.

FIG. 4 is a side view of a portion of an alternative PBR system, wherein an inflatable bladder temporarily/intermittently lifts at least a part of the PBR tube to move settled cells closer to bubbles/turbulence.

FIG. 5 is a transverse cross-section of the embodiment of FIG. 4, viewed along the line 5-5 in FIG. 4.

FIG. 6 is a side view of a portion of an alternative PBR system wherein bands surround the PBR tube in longitudinally-spaced-apart locations, constricting the tube at those locations to increase bubbles/turbulence near settled cells.

FIG. 7 is a transverse cross-section of the embodiment of FIG. 6, viewed along the line 7-7 in FIG. 6.

FIG. 8 is a side view of a portion of an alternative PBR system wherein exemplary block members push down on the PBR tube in longitudinally-spaced-apart locations, changing the tube shape at those locations to force bubbles down toward the tube bottom surface to increase bubbles/turbulence near settled cells.

FIG. 9A is a transverse cross-section of the embodiment of FIG. 8, viewed along the line 9-9 in FIG. 8.

FIG. 9B is a transverse cross-section of an alternative PBR system, showing an exemplary block member provided underneath the PBR tube to change the tube shape at the block location to move the tube bottom surface and settled cells upward closer to the bubbles/turbulence. Blocks may be provided at multiple longitudinally-spaced-apart locations to change the tube shape at the multiple locations.

FIG. 10 is a side view of a portion of an alternative PBR system wherein interior baffles are provided in the PBR tube in longitudinally-spaced-apart locations, reducing/constricting certain areas of the tube diameter to increase bubbles/turbulence near settled cells.

FIG. 11 is a transverse cross-section of the embodiment of FIG. 10, viewed along the line 11-11 in FIG. 10.

FIG. 12 is a head end view of three bioreactors according to an embodiment of the invention, with air, CO2, and cooling water that is used by multiple bioreactors being supplied on a rack above the bioreactors.

FIG. 13 is a head end view of an assembly of nine bioreactors of the embodiment shown in FIG. 12, in three groups spaced apart from each other so that personnel may move between the groups.

FIG. 14 is a side view of an embodiment of the head piece, disconnected from the flexible bioreactor tubing, used in the bioreactor embodiment of FIGS. 12 and 13.

FIG. 15 is a longitudinal cross-sectional view of the head piece of FIG. 14, with only a portion of the air/fill inlet and the CO2 and nutrient ports/lines shown.

FIG. 16 is a side view of a proximal portion of an alternative bioreactor embodiment, using rigid collars to connect tubing portions and to provide ports for sensing probes and sampling devices.

FIG. 17 is a side view of the distal portion of the bioreactor embodiment of FIG. 16, showing an embodiment of the tail piece closing the tail end of the bioreactor except for gas venting equipment.

FIG. 18 is top perspective view of the tail piece of FIG. 17, shown disconnected from the flexible tubing and from the vent line.

FIG. 19 is a longitudinal cross-sectional view of the tail piece of FIGS. 17 and 18, shown connected to the flexible tubing but disconnected from the vent line.

FIG. 20 is a top front (head end) perspective view of multiplexing multiple photobioreactor tubes, specifically in this embodiment six bioreactors in two groups, including an exemplary water-cooling line with nozzles/sprayers for evaporative cooling of the bioreactor(s), and frames for supporting shade tarps or screens in various extends of unrolling or unfurling for controlling the amount of light and/or sunshine on the bioreactors.

FIG. 21 is a graph showing cell growth data over time in a method of cultivating cells according to certain embodiments of the invention, in photobioreactors ICH07A, ICH08A, and ICH09A.

FIG. 22 is a left side, head-end (front), perspective view of multiple photobioreactors according to certain methods of the invention, in use cultivating algae or other cells, and wherein filters and valving are visible above the head pieces of the photobioreactors and shade tarps are visible above the head pieces and extending distally along the length of the photobioreactor tubes.

FIG. 23 is a head-end (front) perspective view of three photobioreactors according to certain embodiments of the invention, wherein each of the flexible tubes of the photobioreactors contains cell suspension being grown in the tubes. The tube at the far left appears dark because of a high cell concentration in the suspension inside that tube, while the tube at the far right appears light because it has been recently started-up for cell growth and/or recently inoculated. In each tube, large bubbles of mixing air are visible as they travel from the head pieces toward the tail end of the photobioreactors.

FIG. 24 is a right side view of an embodiment of a head piece for a photobioreactor, wherein the dark surface at the distal end of the head piece may be coating, wrap, sealant, and/or other treatment to assist is connection of a tube/tube portion to the head piece, for example.

FIG. 25 is a top view of an embodiment of a collar for sensing and sampling at a location along the length of a photobioreactor tube, wherein three sensing probes are installed in three ports and a sampling syringe is installed in a fourth port. The ports are sealed to their respective probes or sampling devices to prevent leaking and contamination of the cell suspension. The sensing probes are adapted to send data to monitoring and/or recording instrumentation, for example, by the electrical cords/cables shown in this view. Alternatively, other means of data transmittal, such as wireless, be used.

### DETAILED DESCRIPTION OF CERTAIN EMBODIMENTS

In the Figures, and in the Appendices to the Specification of the Provisional Application incorporated herein, there are shown several, but not the only, embodiments of the disclosed photobioreactor (PBR) for contained microorganism cultivation, e.g., microalgae cultivation. Certain embodiments of the invention comprise or consist essentially of one or more photobioreactors and/or components/pieces-parts of the photobioreactor(s), certain embodiments of the invention comprise or consist essentially of methods of using any or all of the photobioreactors and/or the components/pieces-parts for cultivation of algae, or other cells as disclosed later in this document, for example, and/or certain embodiment comprise or consist essentially of cells produced, cultivated, or cultured by any of said photobioreactors, components/piece-parts, and/or methods. Certain embodiments of the apparatus and/or methods provide or feature one or more of the following: effective mixing in an enclosed PBR; excellent gas/liquid mixing; no moving parts; no moving parts inside the PBR bioreactor tube; no moving parts inside or connected to the PBR bioreactor tube except connection to an air fan and connection to a nutrient injection pump; a PBR that is very inexpensive; a PBR that can be used to grow GMO microalgae outdoors; and/or a PBR that provides flexible and adaptable operations for a variety of applications. Certain embodiments of the tubular PBR are constructed of polyethylene film and PVC, making it inexpensive. Certain embodiments of the PBR are nearly horizontal, but do not exhibit plug flow. Instead, internal waves propagate the length of the PBR providing enhanced turbulence and gas exchange. Certain embodiments of the PBR do not require, and do not comprise, pumps to circulate the culture; a low pressure/high volume blower is used to provide air and CO₂ and to generate the internal waves. Because certain embodiments of the PBR are constructed with inexpensive polyethylene, the tube/tubing portions that serve as the solar collector for cell growth can be replaced if the tube/tubing portions become too heavily encrusted with biofilm.

Certain embodiments solve these problems with a PBR system that is inexpensive to construct and to operate, even when many independent or substantially-independent PBR's are built. In certain embodiments, “substantially-independent” means that there may be a common air supply fan, common CO2 supply tanks, and/or common cooling water supply into multiple bioreactor tubes (each having separate control and cleaning/filtration for each tube), but that the multiple bioreactor tubes including their head and tail pieces are otherwise separate and independent of each other and the cell suspension inside each bioreactor tube is kept separate from the cell suspensions of all other of the bioreactor tubes. For example, the inexpensive construction and operation is achieved by one or more of the following:

a. nearly-horizontal bioreactor tubes, with resulting close-to-the-ground support structure and easy access to each of the bioreactor tubes from the ground by construction and operating personnel;

b. low-cost and easily-replaceable bioreactor wall(s) in the form of flexible polymeric tubing;

c. no moving parts inside the bioreactor, with resulting low maintenance and low downtown;

d. use of few or no liquid pumps, few or no cell suspension pumps;

e. no moving parts inside the PBR bioreactor tube, or no moving parts inside or connected to the PBR bioreactor tube except connection to an air fan and connection to a nutrient injection pump

f. low pressure air fans rather than high pressure/compressors, with resulting low initial investment and low ongoing energy costs;

g. modular construction and operation approach, so that multiple PBRs can be separately or substantially-separately, and differently, operated for growing and optimizing multiple different species/strains, with little or no chance of cross-contamination between the PBRs, and with different start-up and shut-down schedules possible for each PBR.

In some preferred embodiments, elongated main body of the PBR is comprised essentially of flexible plastic tubing, for example 10 mil polyethylene tubing or other transparent tubing, set on a surface with a slight inclination. Transparency is preferred so that an operator of the bioreactor may see the cell suspension and the conditions inside the tubing. For example, the main body may comprise a single elongated flexible plastic tube, or multiple elongated plastic tube portions that are connected together end-to-end to form the elongated main body. When full of water/culture, each flexible plastic tube or tube portion looks like a rigid or substantially-rigid transparent pipe. The terms “tube” and “pipe” are used herein for both embodiments comprising a single elongated flexible plastic tube and embodiments comprising multiple elongated plastic tube portions connected together end-to-end. The tubing may be selected from inexpensive, flexible plastic tubing that is hollow and cylindrical, but in certain embodiments, the terms “tube” and “pipe” and the described bioreactor main body may include cross-sectional shapes other than circular.

In some embodiments, the elongated body of the photobioreactor (PBR) comprises ABS (acrylonitrile butadiene styrene), CPVC (post chlorinated polyvinyl chloride), PB-1 (polybutylene), PP (polypropylene), PVDF (polyvinylidene fluoride), PE-RT (polyethylene RT), PEX (cross-linked polyethylene), polycarbonate tubing.

At the lower end of the tube, low-pressure air, for example<10 psig air, is introduced into the culture. This air is produced by a high volume, low pressure air blower. The air inside the tube takes the form of a substantially-constant flow of very large bubbles, which are at least several inches in diameter and which travel up-slope along the tube. As they do so, the gas/liquid interface takes the shape of multiple waves that travel up the tube, inducing good turbulence and consequently good mixing and gas exchange.

The PBR tube may be of various cross-section sizes and various lengths. In some embodiments, the tube may be from about 1-60 inches in diameter, and about 50 to 500 feet in length, for example, the tube can be about 1, 2, 3, 4, 5, 6, 10, 15, 20, 25, 30, 35, 40, 45, 50, 55, or 60 inches in diameter and about 50, 60, 70, 80, 90, 100, 125, 130, 140, 150, 160, 170, 180, 190, 200, 225, 250, 275, 300, 325, 350, 400, 450, or 500 feet in length. In some embodiments, the bioreactor tube diameters are about 6 inches up to 12 inches, and about 200 feet long.

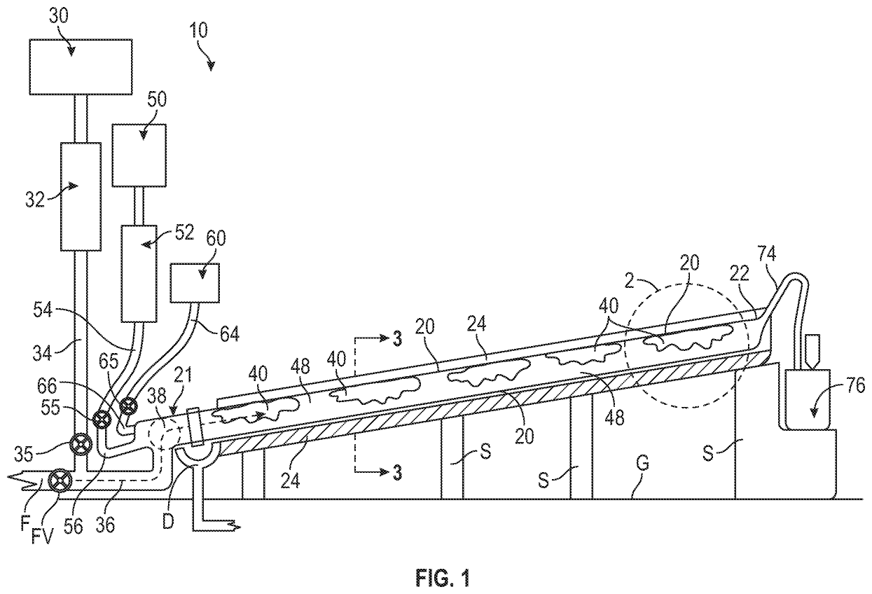

Referring specifically to schematic FIGS. 1 through 3, there is shown apparatus and operation of certain embodiments of a single bioreactor tube of constant or generally constant tube diameter, without baffle, diameter-reduction, or tube-movement adaptations for aiding in cell suspension. It will be understood that FIGS. 1-3 may represent operation of a single tube, but the structure and operation therein may be multiplied, by placing and operating multiple, parallel bioreactor tubes side-by-side for cultivating a greater volume of a single microalgae, or for cultivating different microalgae. As discussed above, the multiple parallel bioreactor tubes may be operated separately and independently, or substantially separately and independently, with common but separately controlled and cleaned/filtered air and CO2 and cooling water supplies.

FIG. 1 portrays PBR system 10 according to certain embodiments of the disclosed technology. The PBR system 10 includes a flexible bioreactor tube 20 resting in a curved or generally-U-shaped support trough 24, which is shown in cross-section in FIG. 1 to allow the viewer to see the tube 20 and the bubbles 40 and suspension 48 in the interior space 28 of the tube 20. Support columns S hold the trough 24 up off the ground G. The support columns are of varying lengths (heights above the ground G) to hold the trough 24, and consequently the tube 20, at a slight slope relative to horizontal and the ground G, all along the length of the tube 20. Tube 20 has at its lower end a head piece 21, and at it upper end a tail piece or “tail” 22. Various amounts of slope may be used, with currently-preferred amounts being in the range of 1 degree up to 3 degrees from horizontal. In certain embodiments, the tube 20 near the head piece and near the tail slopes at 1 degrees to horizontal (about 1.7 percent slope), and the middle portion between the head piece and tail slopes at 2.5 degrees to horizontal (about 4.3 percentage slope). In certain embodiments, the trough and tube slope at a constant amount, for example, 1 degree, 2 degrees, or 3 degrees.

The head piece 21 may be, for example, a rigid, generally cylindrical end-piece that is connected and sealed at its distal end to tube 20. The interior space 38 of head piece 21 is in fluid communication with the hollow interior space 28 of flexible tube 20, so that liquid or gas supplied to the interior space 38 may flow to the interior space 28 of the tube 20. Initial startup of the bioreactor system may comprise filling the tube 20 with water supplied through fill/harvest line F, controlled by fill/harvest valve FV, and sterilization of the water and interior space 28 of the tube 20 by UV or ozone, for example. Ozone sterilization may be done by ozone injection through one or more gas lines into the head piece 21 or into other apparatus in fluid communication with the tube interior space 28, as will be understood by those of skill in the art. At least a portion of the head piece 21, and its connection to the flexible plastic tube 20, is located over a drain trough D that flows to a sewer or other waste treatment. Trough D is provided for catching leaks or drainage that may occur at various/any location along the length of the tube 20, as may occur during start-up, shut-down, or maintenance steps, or due to tube damage, for example.

For ongoing operation of the bioreactor, the proximal end of the head piece 21 is in fluid communication with inlet air, CO2, nutrient, and/or inoculation systems, that may be adjusted and monitored to encourage optimal cell growth in each tube. For example, an air source 30 supplies air, via filtration and control/metering/measurement 32, air line 34, air valve 35, and fill/air inlet 36 (also usable in shut-down as a harvest outlet), to the interior space 38 of the head piece 21, and hence to the interior space 28 of tube 20. CO2 source 50, such as pressurized CO2 tank(s)/canister(s), supplies CO2 required for cell growth, via filtration and control/metering/measurement 52, CO2 line 54, CO2 valve 55 (a shut-off valve, for example), and CO2 inlet 56, also to the interior space 38 of the head piece 21 and hence to the interior space 38 of tube 20. Nutrient source 60 including an adjustable drip/intravenous-type (I.V.) bag/system or other injection and control/metering/measurement, for example, supplies cell nutrients via nutrient line 64, nutrient valve 65, and nutrient inlet 66, and hence to the interior of tube 20. Inoculation of the liquid/culture medium inside the tube 20 may be done during startup using a peristaltic pump or other pump in fluid communication with interior space 38 and hence with the interior space 28 of tube 20, for example, via connection to the nutrient inlet 66. The control, metering, and filtration of water, ozone, air, CO2, nutrients, and inoculant, may be accomplished using commercially-available valves, electronic and/or manual control, flow-meters, and filters, as will be understood from this document and the drawings by those of average skill in this field. The control of each inlet stream may include control valve(s), one-way valves, and shut-off valves, as will be understood from this document and the drawings, as needed to control the inlet flows in a precise and reliable manner.

Operating conditions and culture characteristics may be monitored, for example, by various sensors installed in the head piece 21 or at other locations in the PBR system. For example, pH and/or temperature probes/sensors may be used to monitor and control conditions in the tube 20 or in the environment surrounding the tube. For example, pH probe(s) may be used for adjustment of CO2 addition. For example, temperature probe(s) may be used for cooling the tube 20 and its contents. Said cooling may be conducted if needed, through part or all of the cultivation process, for example, using an evaporative cooling system of water lines and spray nozzles that may extend along substantially the entire length of the tube 20. Condensed cooling water may drain to the tube-support trough 24, and then to drain trough D.

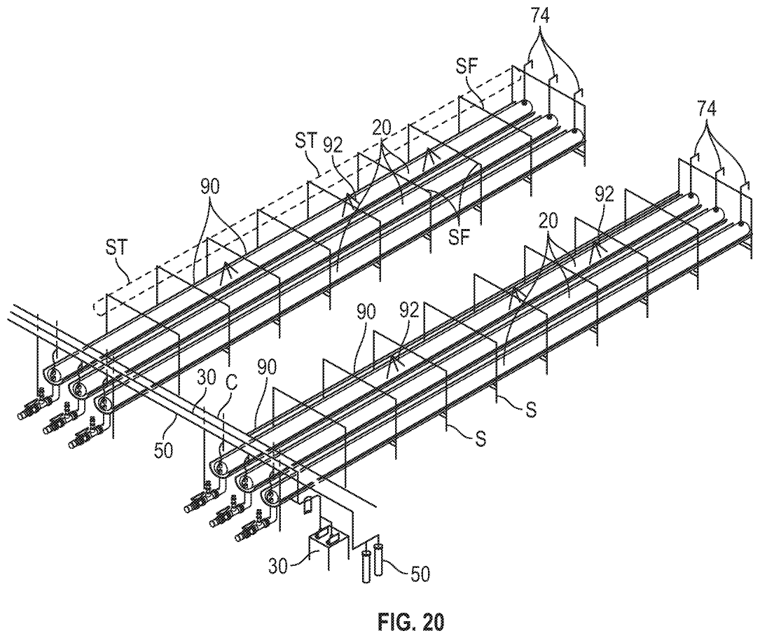

The air source comprises a low-pressure air fan (see fan 30 in FIG. 20), for example a REPUBLIC BLOWER SYSTEMS™ Model HRB401 blower, which can produce a large, low-pressure air flow for one or more bioreactor tubes. This exemplary blower has produced sufficient low-pressure air for large-bubble mixing in twelve bioreactor tubes at the same time. As discussed in the Summary and in the following descriptions of the Figures, such a low-pressure air fan provides air volume and bubble size and frequency that produce excellent mixing and gas-liquid exchange, with a low initial investment and low maintenance cost.

As shown in FIG. 1, the tail piece 22 may be a generally cylindrical end-piece, collar, or other connector between tube 20 and a gas exit or “vent line” 74. Vent line 74 extends to a bleach trap or other system 76 for neutralizing, scrubbing and/or otherwise cleaning the exhausted vent gasses from the bioreactor. For example, these vent gases may include air, CO2 not utilized by the cells, and oxygen produced by the cells, and in certain cases, entrained liquid and/or cells from the culture/suspension inside the tube 20.

FIG. 1 portrays standard operation of the PBR, after start-up, wherein the interior space 28 of the tube 20 is filled with fluids comprising: liquid suspension 48 (culture comprising water, nutrients, and cells, for example) and gas bubbles 40 (also “air bubbles” or “large bubbles”) comprising air, CO2, and oxygen from the cells. Generally speaking, the low-pressure fan pushes air into interior space 38, against the force of the hydrostatic head of the liquid in the tube 20. The height of the liquid “column” is determined by the difference in the level of the upper end (for example, at tail piece 22) of the tube 20 and the level of the lower end (for example, at the head piece 21). The height of the liquid column may vary, but in many embodiments is several feet, for example, less than 8 feet (so, less than about 3.5 psi of head) or less than 5 feet (so, less than about 2 psi of head).

As illustrated schematically by the dashed circle interior space 38 and the dashed line arrow in FIG. 1, the accumulated air in the interior space 38, upon being pressurized by the air fan to overcome the hydrostatic head, will “pop” or “burp” or “blurp” into the interior space 28 of the tube 20. This tends to occur in a periodic, frequent “popping” or “burping” or “blurping” of large bubbles into the tube 20, and goes on through the operation of the PBR.

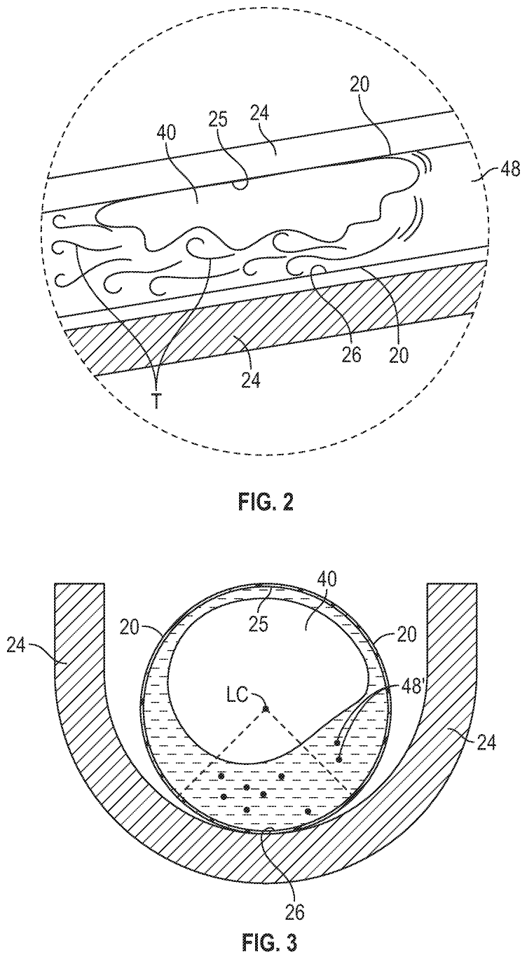

FIGS. 2 and 3 schematically portray details of FIG. 1, to illustrate turbulence T created at and around the large bubble 40 by the bubble movement through the suspension 48, which as schematically portrayed in FIG. 3 comprises microorganisms/cells 48′. In certain embodiments, such as in FIGS. 2 and 3, wherein the tube is generally a constant diameter and no baffles or other flow control/diversion is added to the tube, the large bubbles 40 will tend to float and flow along the top/top surface 25 of the tube 20, due to being a gaseous composition in a liquid suspension 48. However, the bubble movement creates turbulence T that may reach to the bottom surface 26 of the tube 20 in at least some portions of the tube length. Therefore, these large bubbles 40 have been found to be effective in mixing and gas exchange for many species/strains of microalgae and many culture/suspension compositions, as demonstrated by the growth data for certain embodiments in the Appendices to the Specification of the Provisional Application incorporated herein. The terms “bottom 26” and “bottom surface 26” refer to a lower portion of the interior surface of the tube 20 where cells may tend to settle due to gravity, for example, the lower portion of said interior surface extending along the length of the tube 20 and represented by the lower 90 degrees of the circumference of the tube 20, as schematically represented in FIG. 3.

In certain embodiments using a flexible tube 20 of constant 6 inch diameter with no baffle, diameter-reduction, or tube-movement adaptations, the following air flow characteristics have been measured:
a) 3-4 SCFM (standard cubic feet per minute) of air flow per tube 20;
b) 0.5-1 bubble per second flowing from the head piece into the tube 20;
c) bubble velocity at beginning of travel through the tube 20 (soon after leaving the head piece) is about 0.77 meters/second, and near end of travel through the tube 20 (approaching the tail) is about 0.61 meters/second;
d) high amount of turbulence in the tube, wherein some large bubbles coalesce, break apart, or break apart and reform;
e) bubble dimensions have been observed to be very irregular, non-spherical, and non-cylindrical, but average bubble dimension from top to bottom may be in the range of 0.5-1.5 inches, for example; and
f) bubble dimensions have been observed to be very irregular, non-spherical, and non-cylindrical, but bubble length may be from 2 inches up to about 3 feet, for example, and in certain embodiments, 6 inches to 12 inches.
The above-listed flow characteristics a) through f) describe an operation with large bubbles that, compared to the bubbles portrayed in FIG. 1: 1) are longitudinally closer together; 2) become connected (coalesce) as they travel through the tube, break up, and/or break apart and reform; and/or 3) are smaller in diameter relative to the tube and fill less of the tube radial/transverse cross-sectional area. Therefore, while the schematic figures portray certain embodiments, it may be noted that not all embodiments comprise mixing bubbles that are separate and distanced from one another as in FIG. 1, fill a substantial amount of the tube diameter, and remain substantially unchanged in size or shape along the length of the tube.

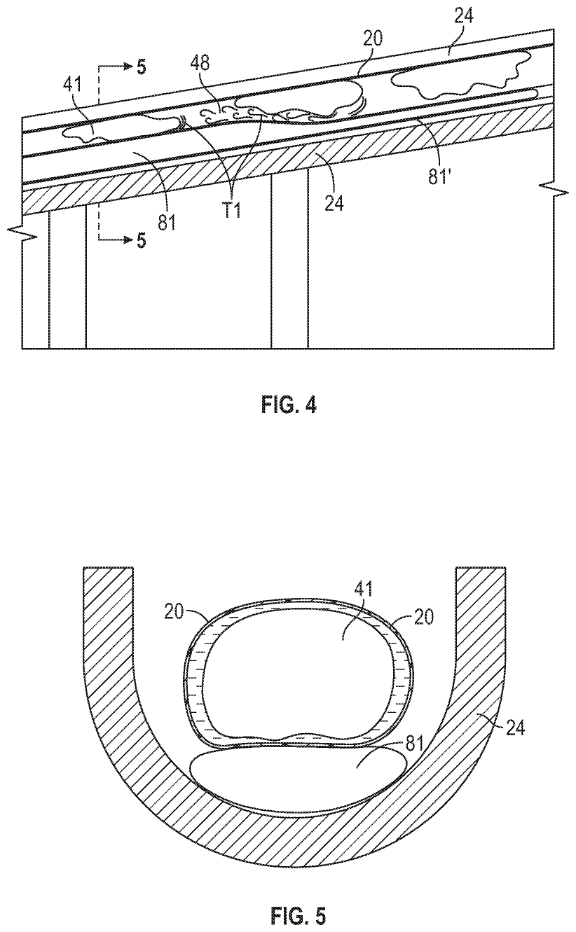

Adaptations may be desirable for increased mixing near the bottom 26, for example, for microalgae that tend to fall out of suspension. Such adaptations may comprise, for example: a) mechanical movement of the tubular bioreactor to move settled cells up off the bottom 26, by lifting and/or increasing/changing the slope of the tube or the bottom tube wall in at least one region; b) reducing the tube diameter at one or more regions to decrease tube diameter relative to bubble diameter; c) changing the tube shape at one or more regions to decrease tube diameter and/or otherwise force bubbles down toward the bottom 26, in effect, creating a baffle in said one or more regions by external force on the outside surface of the flexible tube 20; and/or d) OEM manufacturing or inserting of baffles inside the interior of the tubular bioreactor. Certain of these adaptations may be described as preventing cell settling, moving settled cells from lower-turbulence zones toward higher-turbulence zones, moving turbulence toward settled cells, and/or increasing turbulence. Several of these adaptations will be especially convenient and effective in view of the flexibility of certain embodiments of the tube wall. FIGS. 4-11 schematically portray certain embodiments of these adaptations and the general effect on bubble shape and location, and, hence, on mixing and cell suspension.

FIGS. 4 and 5 illustrate an air bladder 81, 81′ as a means for temporarily/intermittently lifting, and/or changing the slope of, at least one region of the bottom wall of the tube 20. The bladder is being inflated from an air-inlet (not shown in FIG. 4) at the left of FIG. 4, wherein said inflation has increased the height of the bladder 81 and raised/tilted at least the tube 20 bottom wall in that region. Further, as the bladder inflates, the bladder becomes sloped from the inflated portion 81 to the uninflated portion 81′, and this slope may affect the slope of the bottom wall. These changes lift and disturb the cells settled on the bottom 26. Further, the bottom 26 will be closer to the bubble 41 and associated turbulence T1, mixing the settled cells into suspension for improved access to nutrients and CO2, for better and more uniform cell growth throughout the tube 20. It will be understood from FIGS. 4 and 5 and the above discussion that other mechanical means may be used to temporarily/intermittently lift/change the location and/or slope of the tube 20, for example, a rocker system(s), arm(s) intermittently actuated to lift or push regions of the tube 20; and/or a moving rod(s) that slides longitudinally between the tube 20 and trough 24 to create a “rippling” movement under the tube 20 to urge settled cells toward the mixing bubbles and turbulence. The frequency, pattern, and/or extent of said lifting/changing may be adjusted by the tendency of cells to settle, for example, and may be manually actuated by personnel and/or automatically actuated by conventional controllers/programming.

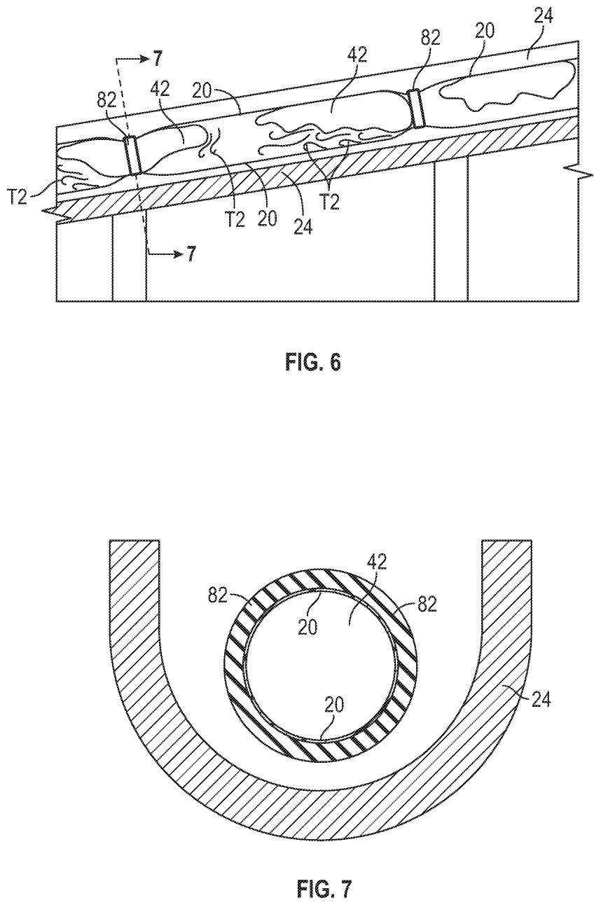

FIGS. 6 and 7 illustrate external constrictions in multiple regions of the tube 20, by bands 82 encircling the flexible tube 20. These bands may be elastic bands, for example, that squeeze the tube in said multiple regions to reduce the tube diameter all the way around the tube circumference. This approach may have multiple benefits, for example, lifting and changing the slope of the flexible bottom tube wall in the region of the band 82, and forcing bubbles 42 through the reduced diameter portion of the tube. As a result, the bubbles 42 and turbulence T2 are closer to the settled cells, and the bubbles 42 may create more turbulence T2 due to the their flow through the reduced diameter of the tube 20 creating a “baffle region” in the tube 20, and mixing and cell suspension are consequently improved.

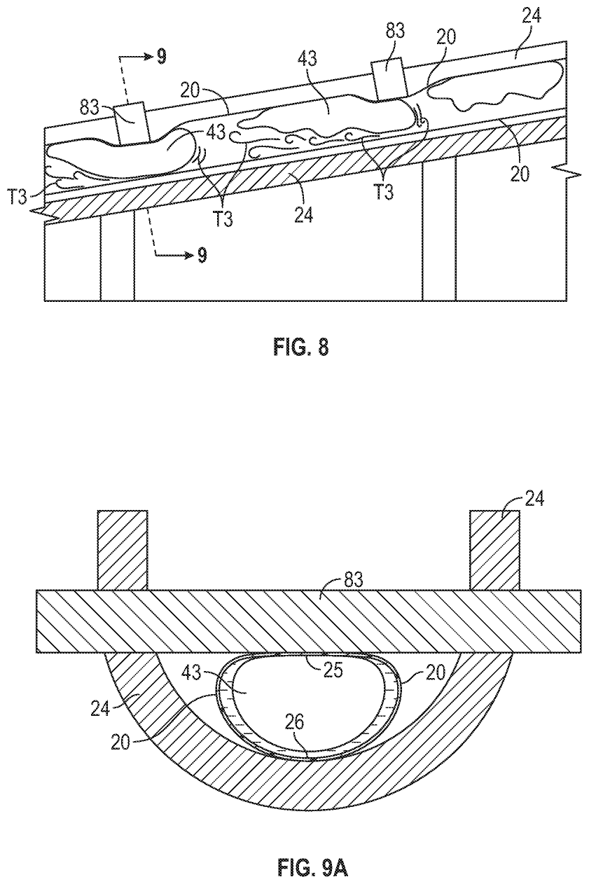

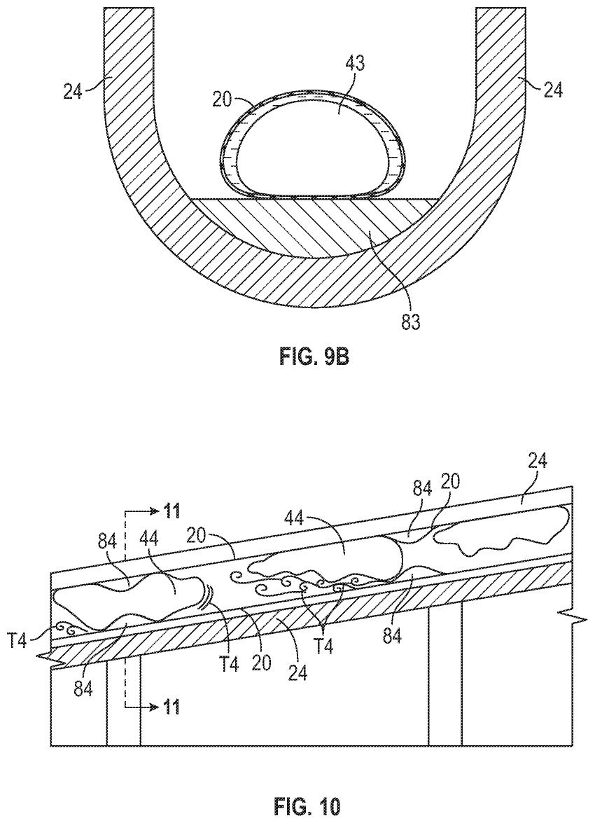

FIGS. 8 and 9A illustrate members 83 pressing down on the top 25 of the tube 20, lowering the flexible top/top surface 25 in certain regions causing constrictions along the length of the tube and to urge bubbles 43 and turbulence T3 toward the bottom 26 of the tube 20 and the settled cells. The members 83 may apply continuous force on the tube top 25, for example, by resting in notches formed in the top edge of the trough 24, or may apply force occasionally or periodically by being moved by mechanical systems or personnel according to a schedule or observation of the need for better suspension. This approach forces the bubbles 43 through the reduced diameter portions (constrictions) of the tube, so that bubbles 43 and turbulence T3 are closer to the settled cells, and the bubbles 42 may create more turbulence T3 due the reduced diameter region creating a “a baffle region” in the tube 20, and mixing and cell suspension are consequently improved.

FIG. 9B illustrates a member 83 pressing provided under the tube 20, which raises the flexible bottom/bottom surface 26 and the settled cells closer to the bubbles 43 and turbulence. Members 83 may be provided at multiple spaced-apart locations under the tube 20, and may optionally be occasionally or periodically moved to various other locations along the length of tube 20, by mechanical systems or personnel according to a schedule or observation of the need for better suspension. This approach forces the settled cells closer to the bubbles 43 and turbulence, and may create more turbulence due to the reduced diameter region creating a “a baffle region” in the tube 20, both of which effects improving mixing and cell suspension. In some embodiments, member 83 comprises multiple ridges, for example, a ridged top surface of the member 83, pressing against the bottom of tube 20, wherein the ridges extend transverse to the longitudinal axis of the tube 20.

FIGS. 10 and 11 illustrate baffles 84 provided on the inner surface of the tube 20 and/or inserted into the tube 20. Baffles 84 create reduced-diameter regions, so that bubbles 44 and turbulence T4 reach closer to at least portions of the interior surface of the tube 20. This approach forces the bubbles 43 through the regions that may be described as reduced diameter, due to their oval (rather than circular cross-sectional) shape, and the consequent reduced dimension from inner top to inner bottom of the reduced-diameter region. Bubbles 44 and turbulence T4 are closer to the settled cells, and the bubbles 43 may create more turbulence T3 due to passing through the reduced-diameter region, both of which effects improving mixing and cell suspension.

Certain embodiments may include various interior baffles or other protrusions having various shapes and locations may be provided as part of the tube wall structure and/or as inserts into the tube, wherein the baffles preferably are shaped and located to increase turbulence to reduce or prevent settling of cells, while not creating low-flow zones or corners in which cells are likely to settle. In certain embodiments, baffles or other protrusions extending toward the longitudinal centerline LC (FIG. 3) of the tube may comprise, consist essentially of, or consist of transverse baffles/protrusions provided on the interior top surface 25, the bottom surface 26, and/or the left or right side surfaces of the tube, in order to increase turbulence and/or control or direct the flow of the large bubbles. For example, certain embodiments may include numerous/many baffles or other protrusions extending toward the longitudinal centerline LC of the tube that are smaller in both radial and axial dimensions compared to baffles 84 in FIGS. 10 and 11. Said baffles/protrusions may include ribs provided in the interior longitudinal bottom surface 26, either provided OEM during manufacture of the tube wall, or as added/inserted structure after manufacture of the tube wall. For example, the ribs may be in the range of 0.1 to 3 inches, 0.1-2 inches, 0.1-1 inches, 0.1 to 0.5 inches, or 0.1-0.25 inches in axial length. For example, the ribs may extend radially from the inner tube wall surface a distance in the range of 1-25 percent, 1-20 percent, 1-10 percent, 1-5 percent, or 1-2 percent of the diameter of the tube 20.

Appendices A, B, C (1-4) and D, of the Provisional Application incorporated herein, provide additional details and information about cultivation and cell growth, construction, piping and instrument layout, and operating procedures for certain exemplary embodiments of the PBR system. Appendix A, as mentioned earlier in this document, provides cell growth for exemplary PBR systems. Appendix B provides piping and instrument diagrams for exemplary PBR systems. Appendix C (in portions 1-4) provides photos of exemplary PBR systems. Appendix D provides Standard Operating Procedures (SOP) for exemplary PBR systems.

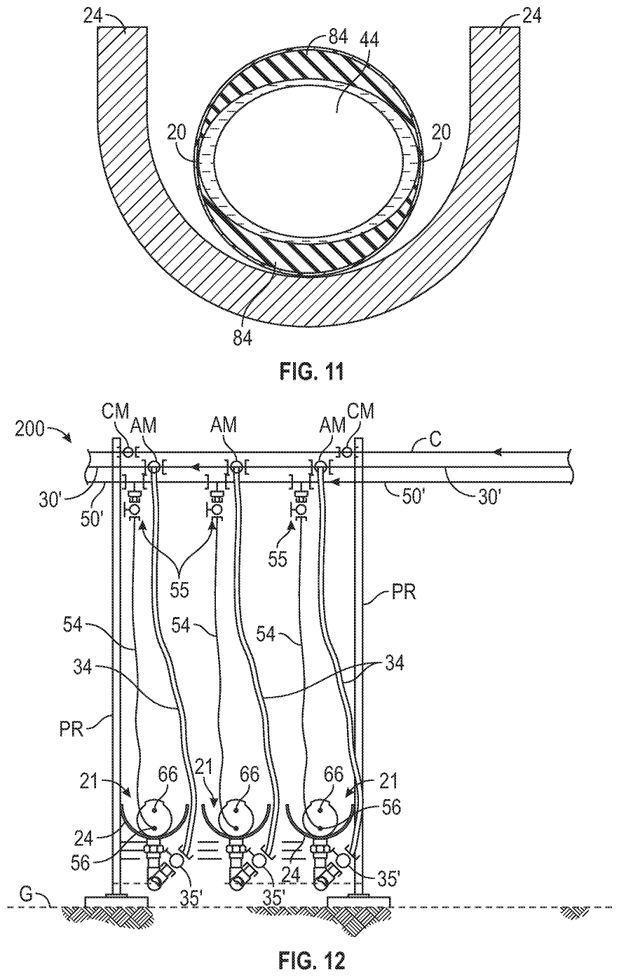

FIG. 12 schematically illustrates an assembly of bioreactors, specifically bioreactor system 200, which is an embodiment comprising at least three bioreactors that are parallel, side-by-side, and supported generally horizontally (extending into the page in FIG. 12). FIG. 12 shows the head ends of the three bioreactors each with a head piece 21, and each having a tube support trough 24, an air system comprising an air line 34 from an air manifold AM, an air valve 35′ at/near the head piece 21, a CO2 system comprising CO2 line 54, a CO2 valve 55, and a CO2 inlet 56 to the head piece, and a nutrient inlet 66 at the head piece (nutrient line not shown in FIG. 12). Behind each head piece 21 in FIG. 12 is a flexible tube 20 for containing a cell suspension, as discussed above. Lines/equipment common to the three bioreactors, and optionally also common to other similar or identical bioreactors that may be provided to the right and/or left of the bioreactors shown in FIG. 12, comprise air source line 30′ from an air source 30 (not shown in FIG. 12), CO2 line 50′ from a CO2 source 50 (not shown in FIG. 12), and cooling water supply lines C. These common lines/equipment are preferably supported on pipe racks PR that extend up along and overhead the head end of groups of three bioreactors, for example, and preferably extend to the adjacent groups of similar or identical bioreactors at the right or left of the three bioreactors shown. Cooling water supply line C may also be common to the three bioreactors and said adjacent groups of bioreactors, and is preferably supported by said pipe racks PR. The cooling water supply line C may comprise manifolds CM that connect to water lines 90 and nozzles 92 (not shown in FIG. 12, but shown in FIG. 20) extending along the length of each bioreactor tube. The three bioreactors shown in FIG. 12 do not comprise any fluid connection that the allows cell suspension inside each bioreactor/tube to flow to or reach any other of the bioreactor/tubes.

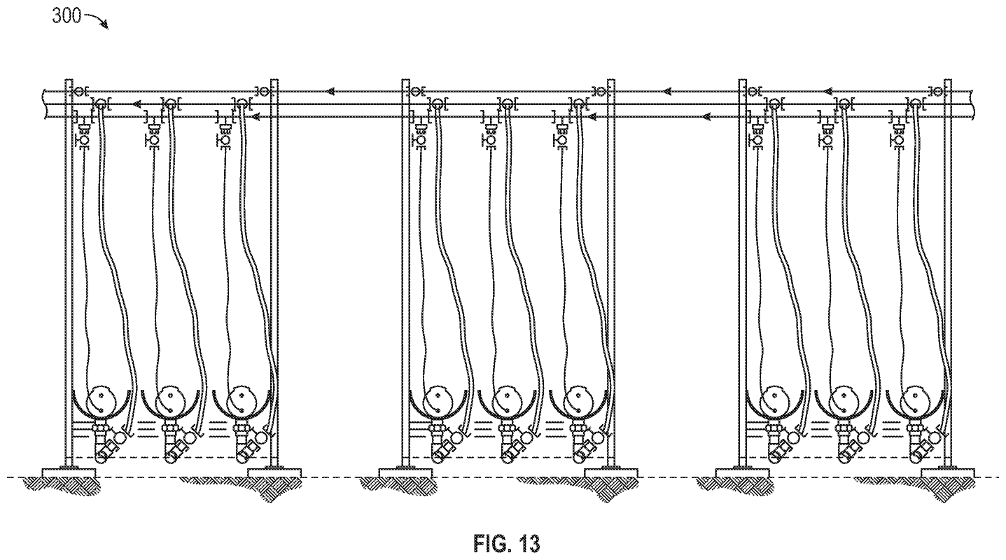

FIG. 13 illustrates a bioreactor assembly 300 of three groups of three bioreactors, wherein each of the three groups may be an embodiment such as portrayed in FIG. 12. Each group of bioreactors has a pipe rack extending up and over the head end of the respective group, to support the air source line, CO2 source line, and cooling water source line (30′, 50′, and C, respectively, in FIG. 12) that extend over and between the three groups of bioreactors. While the air source line 30′, CO2 source line 50′, and cooling water source line C and the preferred fan, CO2 cylinder(s), and cooling water tank/source upstream of these lines 30′, 50′, and C, may be described as common to all the bioreactors, it will be understood from this document that, downstream of the manifolds/connections from these lines to the individual bioreactors, there are control and control valves, metering, filtering, one-way valving, and/or sensing and sampling for each individual bioreactor that adapt each individual bioreactor to operate independently and to prevent contamination from one bioreactor to another. Therefore, the multi-tube embodiment in FIG. 13 comprises at least nine side-by-side and parallel bioreactors, in at leave three groups, each bioreactor being configured so that the cell suspension inside each bioreactor tube cannot flow to or reach any other of the bioreactor tubes. Thus, the embodiment in FIG. 13 is adapted for at least nine separate or substantially separate cell growth operations.

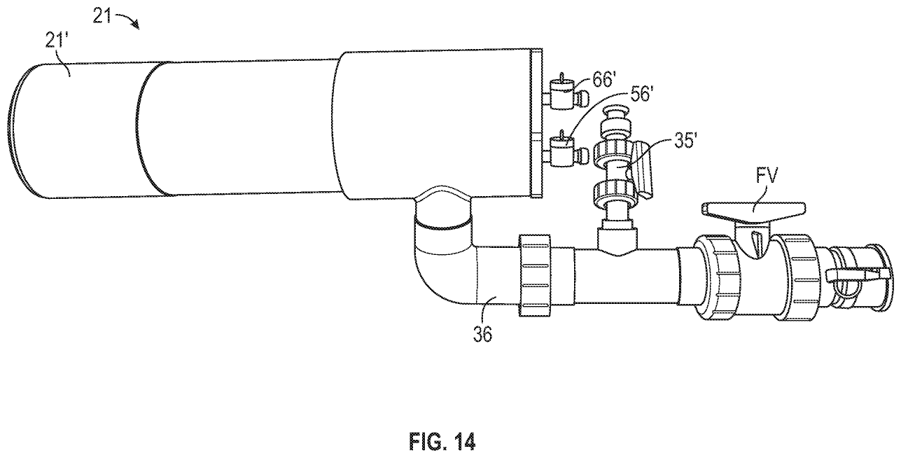

FIG. 14 illustrates an embodiment of a preferred head piece 21 that may be used in each of the bioreactors of FIGS. 12 and 13, for example. The head piece 21 comprises a reduced-diameter distal portion 21′ for connection and liquid sealing to the flexible tube 20 of the respective bioreactor. The head piece 21 comprises at/near its proximal end an air valve 35′ in the air line connected to the fill/air inlet line 36, a fill/harvest valve FV in the fill/harvest line also connected to the fill/air inlet line 36, a valved port 56′ connecting to and/or serving as the CO2 inlet, and a valved port 66′ connecting to and/or serving as the nutrient inlet. The end of the fill/harvest line F, proximal of valve FV, comprises a quick-connector or other connector for detachable connection to, for example: 1) a liquid line for filling the head piece 21 and the tube 20 connected to its distal end 21′, for example, with pre-sanitizing chemicals and/or other liquids as may be needed prior to startup and/or water needed for startup and for the cell suspension, and 2) a harvest or shutdown line for harvesting cell suspension and/or otherwise draining the head piece and tube 20.

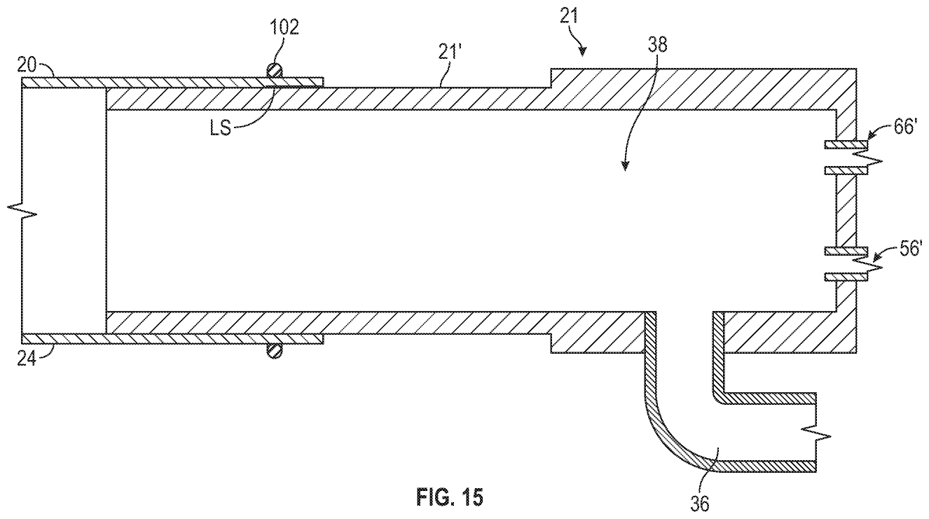

FIG. 15 is a longitudinal cross-section of the head piece 21, showing the distal portion 21′ being connected to tube 20 by band 102 that schematically represents one or more fasteners tightly circling the tube end to secure the tube 20 all around the circumference of the tube 20 to the distal end 21′. The band/fastener(s) may comprise a sealant and/or a sealant LS may be added between, over, or otherwise in contact with, the tube 20 and the distal portion 21′ to prevent fluid, including liquid and gas, from leaking out from between seal tube 20 and the distal portion 21′ . The head piece 21 is open at its distal end for unrestricted fluid communication with the interior of the tube 20. The head piece 21 may be made mainly of a cylindrical, hollow tube, for example, a rigid PVC pipe or other rigid pipe including opaque pipe. FIG. 15 also illustrates that the interior space 38 of the head piece 20 including the distal portion 21′, line 36, port 56′ and port 66′ all being open and empty of equipment, including not including sparger plates, nozzles, orifice plates, baffles, protrusions, or other small-bubble-creating equipment. It may be seen that the head piece 20 embodiment of FIG. 15 comprises a hollow cylindrical or generally cylindrical pipe having a thin cylindrical wall relative to the relatively larger open and empty interior space 38 (without said small-bubble-creating equipment) and that has a proximal end plate/wall comprising multiple ports with open and empty interior passages (also without small-bubble-creating equipment). Further, inlet line 36 also has an open and empty interior passage without small-bubble-creating equipment. The diameter of the interior space 38, all along the head piece 21 from the proximal end plate/wall to the distal end of the distal portion 21′, is the same as the inner diameter of the flexible tube 20, or close to the same as the inner diameter of the flexible tube 20. For example, “close to the same as the inner diameter of the flexible tube 20” may mean within +/−10 percent or within +/−15 percent of the inner diameter of the flexible tube 20, so that the transition between the distal portion 21′ and the tube 20 interior space is not a significant change in diameter of the interior passage through the bioreactor and does not cause any, or any significant, creation of small bubbles.

Certain embodiments of the bioreactor tube 20 may comprise rigid portions, for example, rigid tubes or collars, at one or more locations along the length of the tube 20, including rigid portions that are distal of the preferably-rigid head piece and proximal of the preferably-rigid tail piece. Said rigid portion(s) are connected or fixed to the tube or between tube portions, so that one or more apertures/ports in said rigid portion may be used to gain access to the interior space of the tube 20, for example, for sensing conditions or compositions of, or sampling, the cell suspension in the bioreactor. The rigidity of the rigid portion(s) is important in many embodiments so that sensing probes, sampling devices, or other devices can be installed in the apertures/ports and effectively used with the rigid portion(s) to study the contents of the flexible tube(s)/bioreactor, for example, with durability, consistent operability, and without tearing the tube or tube portions to cause leaking or contamination of the cell suspension. The rigidity allows effective and durable insertion and sealing of the probes, sampling, or other devices into/through the port to prevent leakage and contamination, and insertion through both the port and the tube wall if necessary (when the tube 20 is continuous through/under the rigid portion). The rigidity allows the preferred repeated use of a given rigid portion, for gathering data or samples over an entire cell growth run and preferably over many runs. The rigid portion(s) may be connected or fixed to the tube 20 or portions of the tube 20, by adhesive, epoxy, fasteners, bands, or other securement systems. The rigid portion(s) may encircle the tube 20, and may be moved to various locations and at least temporarily connected or fixed in each of the various locations, for said sensing or sampling, by insertion of the probes/samplers through both the rigid portion's wall and the tube wall, into the hollow interior of the tube. Alternatively, the tube 20 may be cut or otherwise separated into portions of tube 20, which tube portions 20 may be connected to ends of each rigid portion, so that the tubing does not extend through, or at least not entirely through the hollow interior passage of the rigid portion; in such embodiments, the interior passage through the rigid portion fluidly communicates with the interior spaces of the tube portions and, together, the interior passage of the rigid portion, and the interior spaces of the tube portion at each end of the rigid portion, form a single interior passageway through the bioreactor that receives the cell suspension for cell growth, and receives the bubbles flowing from the head end to the tail end of the bioreactor. To provide said interior passage of the rigid portion, the rigid portion is preferably hollow, for example, a cylindrical or generally cylindrical pipe with an interior diameter the same or about the same as the interior diameter of the tube/tube portions.

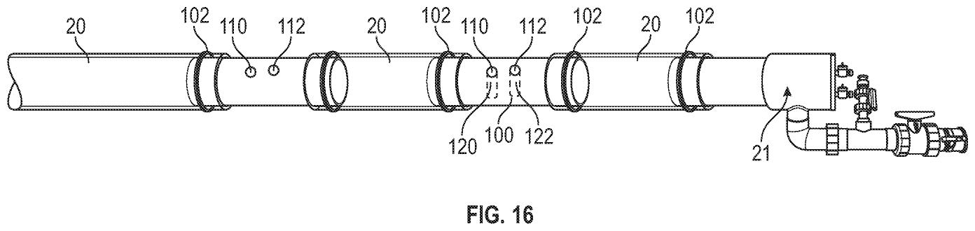

FIG. 16 is a side view of a proximal portion of a bioreactor embodiment that uses the head piece 21 of FIGS. 14 and 15, and multiple portions of flexible tubing 20 connected by an embodiment of said rigid portion(s), that is, multiple collar connectors (“collars”) 100. Each collar 100 is a cylindrical, hollow tube, for example, a rigid PVC pipe or other rigid pipe including opaque pipe, having two open ends to which two of the portions of flexible tubing 20 are fastened and liquid-sealed. Multiple collars 100 are provided at spaced intervals along the length of the bioreactor tube 20 between the head piece 21 and the tail piece 22. Bands 102 are shown to schematically represent the fastening and sealing of the tubing portions all around their circumferences to the collars 100. The collars 100 are provided with one or more ports for sensing of conditions or compositions inside the bioreactor, and/or for sampling small amounts of the cell suspension, at the various locations of the collars and ports. For example, port 110 may be used for a pH, conductivity, temperature, oxygen, and/or other probes/sensors 120 may be used to monitor and provide signals/data regarding the region of the bioreactor where the collar is located, for example, for controlling air, CO2, and/or nutrient flow into the tube/bioreactor. For example, port 112 may be used for a sampler 122, such as a syringe sampler, to withdraw a small amount of the cell suspension from the region of the bioreactor where the collar is located. Each port and the probe, sensor or sampler installed or inserted therein is sealed so that no leaking can occur out of or into the tube/bioreactor and so that no contamination of the cell suspension takes place. By using multiple collars with probes, sensors, or samplers, spaced at multiple locations along the length of the bioreactor, an operator can monitor and/or sample at said multiple locations for gaining substantial amounts of data, and/or gaining and comparing data from different regions of the bioreactor, for example, from regions near the head end versus near the tail end of the bioreactor. For example, cell growth rates at the various regions may be studied. Collars provide an area of the bioreactor tube to install probes and sample ports along the length of the system. This allow segmenting of the reactor tube in order to determine growth rates along the system as well as to begin implementing various control features. In certain embodiments, probes, sensors, and syringe samplers sealed to/in the collar ports may be adapted from commercially-available equipment. Probes/sensors may be held in place in their respective ports by cord grips and by screw-tapping into the PVC-pipe collar and sealed with epoxy. To provide a syringe sampler. a metal tube attached with tubing and a Luer Lok™ syringe act as a sample port while maintaining sterile conditions in the bioreactor while being able to assess growth rates and any other analyses.

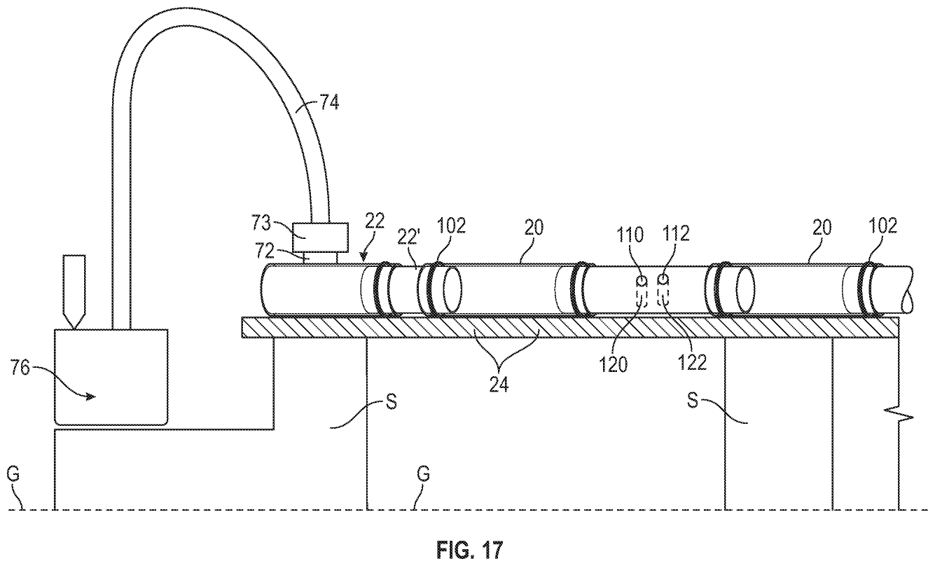

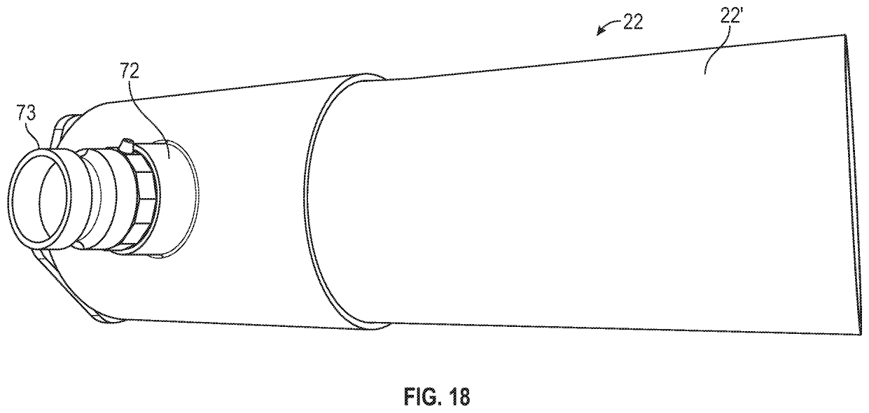

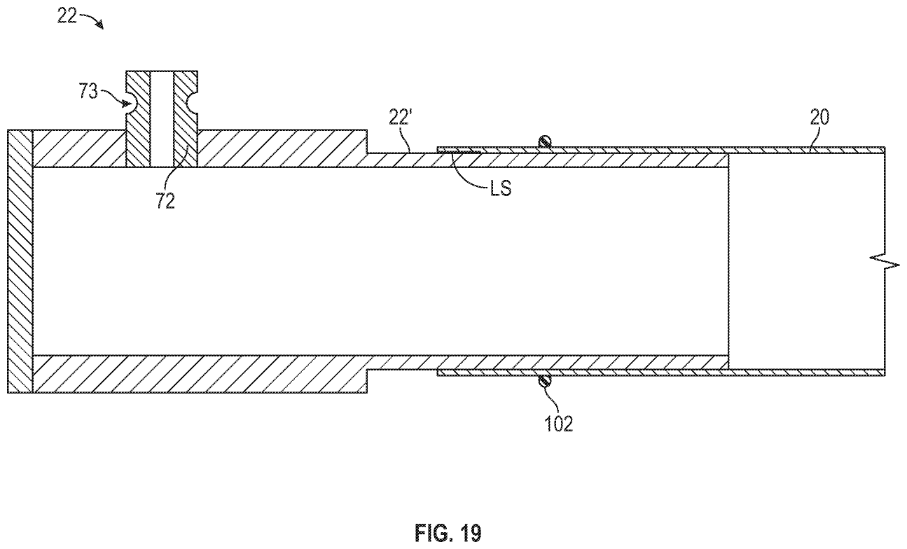

FIG. 17 is a side view of the distal portion of the bioreactor embodiment of FIG. 16, including tubing portions 20, and collars 100 with ports 110, 112, bands 102, and liquid and gas sealant LS between the tube portion 20 and the tail piece, as described above regarding FIG. 16. FIG. 17 portrays the tail end of the bioreactor that comprises tail piece 22 with a vent port 72, vent line connector 73, vent line 74, and bleach trap or other neutralizing/scrubbing system 76 for the gas stream exiting the tail end that may contain entrained or other liquid. The tail piece 22 may be made mainly of a cylindrical, hollow tube, for example, a rigid PVC pipe or other rigid pipe including opaque pipe. The port 72 and connector 73 may be adapted from commercially-available equipment. FIG. 18 provides a top perspective view of the tail piece 22 with its reduced-diameter proximal portion 22′, and the male portion of the connector 73 for the port 72. FIG. 19 provides a longitudinal cross-sectional view of the tail piece 22 of FIGS. 17 and 18, showing that this embodiment is a hollow cylindrical or generally cylindrical pipe having a thin cylindrical wall relative to the relatively larger interior space. The tail piece 22 has a distal end plate/wall without ports, and a proximal opening in its proximal portion 22′ that places the interior space in fluid communication with the interior of the tube 20. Vent port 72, as described above regarding FIGS. 17 and 18, is provided in an upper portion of the tail piece wall. The diameter of the interior space of the tail piece 22, all along the tail piece 22 from the distal end plate/wall to the proximal portion 22′ opening, is the same as the inner diameter of the flexible tube 20, or close to the same as the inner diameter of the flexible tube 20. For example, “close to the same as the inner diameter of the flexible tube 20” may mean within +/−10 percent or within +/−15 percent of the inner diameter of the flexible tube 20, so that the transition between the proximal portion 22′ and the tube 20 interior space is not a significant change in diameter of the interior passage through the bioreactor and does not cause any, or any significant interference with gasses existing the tail end of the bioreactor.

FIG. 20 provides a top perspective view of a bioreactor system comprising multiple bioreactors groups, specifically two groups of three bioreactors with space for a personnel walkway between the groups. It will be understood that many more bioreactors according to the invention may be installed and used, for example any number from 2 up to 100, or any number from 2 up to 200 bioreactors. The bioreactors are parallel and side-by-side to each other, and comprise fluid inlets at the head pieces toward the left of the figure and gas venting at the tail piece toward the right of the figure, as described above. The pipe rack is shown at the head end, supporting air source line, CO2 source line, and a cooling water line. An exemplary cooling water line 90 with cooling water spray nozzles 92 is shown next to one of the bioreactors; additional lines 90 and nozzles 92 will typically be provided, for example, one line 90 with nozzles 92 for each bioreactor for individual, controlled temperature control of each bioreactor. Multiple shade frames SF are provided beside and extending over each group of bioreactors, for use in supporting and adjusting the position, and the extent of coverage, of shade tarps ST or fabrics over one or more of the bioreactors, to further control the temperature and the amount of sunshine and/or light falling on each bioreactor.

Said cooling by cooling water and/or shade tarp(s) may be conducted if, and to an extent, needed, through part or all of the cultivation process. Preferably, water lines 90 and spray nozzles 92 extend from the cooling water line C via cooling water manifolds CM and run along substantially the entire length of the tube 20. The provides an economical and easily-controlled cooling system, using evaporative cooling resulting from the water being sprayed on and adjacent to the bioreactors. Condensed cooling water may drain from the bioreactor to the tube-support trough 24, and then to drain trough D. Shade tarps or simply “shade” ST, which are adjustable to any position including positions fully, partially, or not at all shading the bioreactors, are also an economical and easily-controlled cooling and/or light control system.

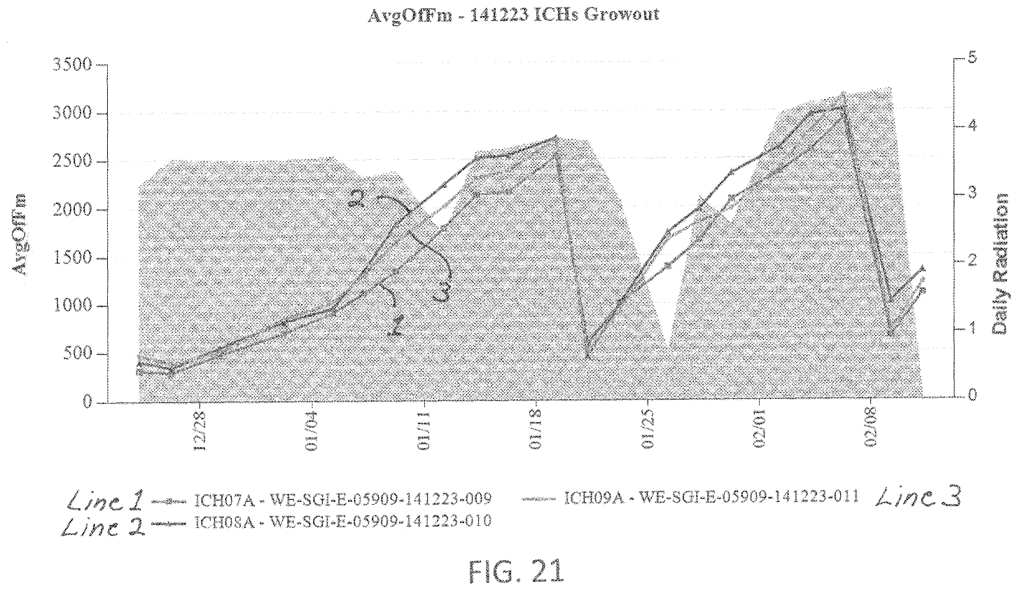

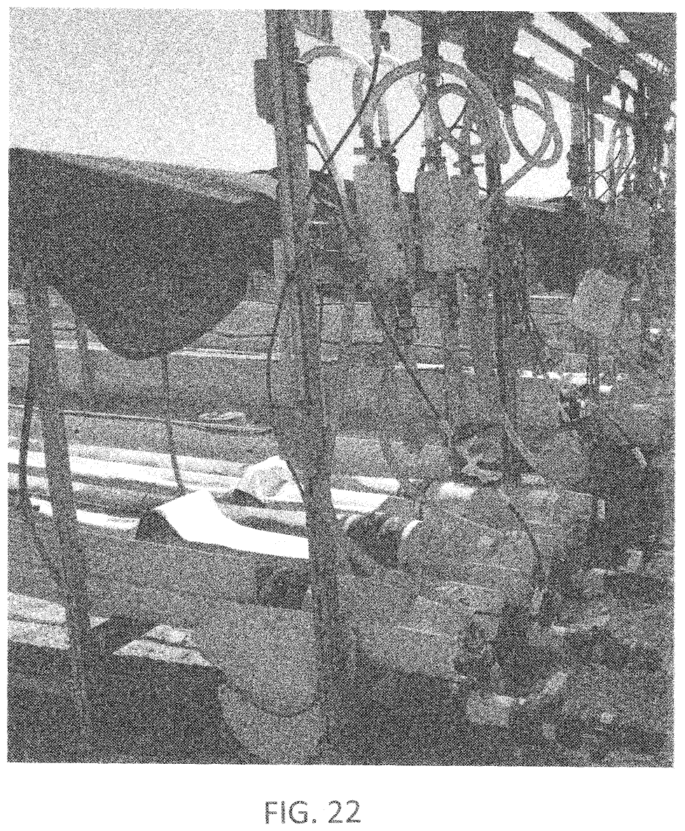

FIG. 21 is a graph showing cell growth data over time in a method of cultivating cells according to certain embodiments of the invention, in photobioreactors ICH07A, ICH08A, and ICH09A.

FIG. 22 is a left side, head-end (front), perspective view of multiple photobioreactors according to certain methods of the invention, in use cultivating algae or other cells, and wherein filters and valving are visible above the head pieces of the photobioreactors and shade tarps are visible above the head pieces and extending distally along the length of the photobioreactor tubes.

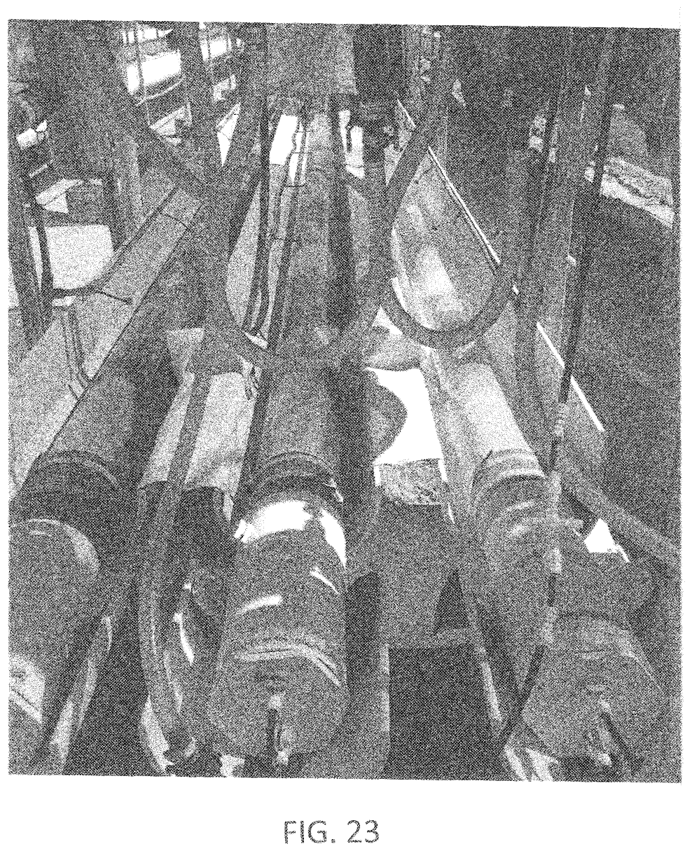

FIG. 23 is a head-end (front) perspective view of three photobioreactors according to certain embodiments of the invention, wherein each of the flexible tubes of the photobioreactors is full, or substantially full in that each is 80-99 or 80-95 volume percent, or more preferably 90-95 volume percent full, for example, of cell suspension being grown in the tubes. It may be noted that, when the tubes are not full of liquid, the flexible tubes may collapse and/or otherwise flex/bend to become non-cylindrical. The tube at the far left appears dark because of a high cell concentration due to the cells having grown a relatively long time compared to those in the middle and far right tubes. The tube at the far right appears light because it has been recently started-up for cell growth and/or recently inoculated, so that the cell concentration is very low. In each tube, large bubbles of mixing air, for example as described earlier in this document, are visible as they travel from the head pieces, through the tubes, toward the tail ends of the photobioreactors.

FIG. 24 is a right side view of an embodiment of a head piece for a photobioreactor, wherein the dark surface at the distal end of the head piece may be coating, wrap, sealant, and/or other treatment to assist is connection of a tube/tube portion to the head piece, for example.

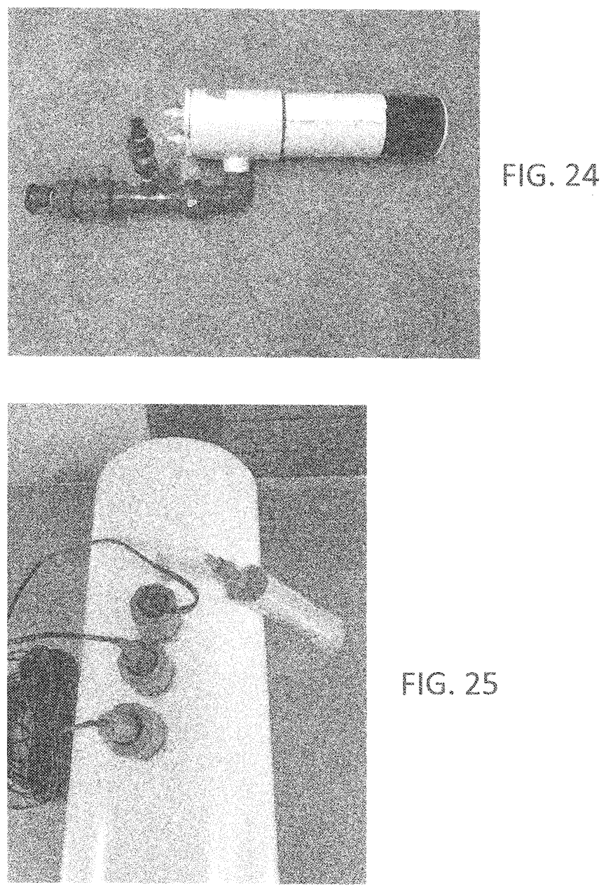

FIG. 25 is a top view of an embodiment of a collar for sensing and sampling at a location along the length of a photobioreactor tube, wherein three sensing probes are installed in three ports and a sampling syringe is installed in a fourth port. The ports are sealed to their respective probes or sampling devices to prevent leaking and contamination of the cell suspension. The sensing probes are adapted to send data to monitoring and/or recording instrumentation, for example, by the electrical cords/cables shown in this view. Alternatively, other means of data transmittal, such as wireless, be used.

In some embodiments, the cell is a photosynthetic microorganism. In some embodiments, the photosynthetic microorganism is a eukaryotic microalga. In some embodiments, the eukaryotic microalga is a species of Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Picochlorum, Platymonas, Pleurochrysis, Pleurococcus, Prototheca, Pseudochlorella, Pseudoneochloris, Pyramimonas, Pyrobotrys, Scenedesmus, Schizochlamydella, Skeletonema, Spyrogyra, Stichococcus, Tetrachorella, Tetraselmis, Thalassiosira, Viridiella, or Volvox.

In some embodiments, the photosynthetic microorganism is a cyanobacterium. In some embodiments, the cyanobacterium is an Acaryochloris, Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, thermosynechocystis, Tolypothrix, Trichodesmium, Tychonema, or Xenococcus species.

In some embodiments, the microorganisms are Chytrid.

As used herein, the terms “about” or “approximately” when referring to any numerical value are intended to mean a value of plus or minus 10% of the stated value. For example, “about 50 degrees C.” (or “approximately 50 degrees C.”) encompasses a range of temperatures from 45 degrees C. to 55 degrees C., inclusive. Similarly, “about 100 mM” (or “approximately 100 mM”) encompasses a range of concentrations from 90 mM to 110 mM, inclusive. All ranges provided within the application are inclusive of the values of the upper and lower ends of the range.

In the Summary of the Invention above, throughout the Detailed Description, and in the accompanying drawings, including of the Provisional Applications incorporated herein, reference is made to particular features, apparatus, and method steps of certain embodiments of the invention. It is to be understood that the disclosure of the invention in this specification includes all possible combinations of such particular features, apparatus, and methods. For example, where a particular feature is disclosed in the context of a particular aspect, a particular embodiment, or a particular Figure, that feature can also be used, to the extent appropriate, in the context of other particular aspects, embodiments, and Figures, and in the invention generally. Although this disclosed technology has been described above with reference to particular means, materials and embodiments, it is to be understood that the disclosed technology is not limited to these disclosed particulars, but extends instead to all equivalents within the broad scope of this disclosure and of the following claims.

1. A photobioreactor system comprising one or more elongated bioreactors, wherein each bioreactor comprising a flexible tube, a head piece connected to head end of the tube and a tail piece connected to a tail end of the tube, each elongated bioreactor being provided at one or more near-horizontal angles relative to the ground, said near-horizontal angles in the range of 1-8 degrees to horizontal, and each bioreactor having an elongated interior space containing cell suspension mixed by an air mixing system;
wherein the air mixing system comprises an air inlet into an interior of the head piece of each bioreactor, the head piece adapted so that air from the air inlet enters and accumulates in the interior of the head piece, until air pressure in the head piece increase to be higher than hydraulic head in the bioreactor and the air in the head piece moves in large bubbles from the head piece into the tube and toward the tail end of the tube; and wherein no cell suspension from any of the elongated bioreactor enters any other of the one or more elongated bioreactors, so that the bioreactors are adapted for growing different cells.

2. The photobioreactor system as in claim 1, wherein the head piece comprises an air inlet, a CO2 inlet, a nutrient inlet, and a line for water input or cell harvesting.

3. The photobioreactor system as in claim 2, wherein all air, CO2, nutrients, and water input into the bioreactor is input into said head piece.

4. The photobioreactor system as in claim 3, wherein each head piece is a rigid pipe closed at a proximal end except for an inlet pipe connected to said air inlet pipe connected to said air inlet and said line for water input or cell harvesting, the CO2 inlet, and the nutrient inlet, and wherein each head piece is open at a distal end for fluid communication with the tube including said large bubbles moving from the head piece to the tube.

5. The photobioreactor system as in claim 4, wherein the head piece comprises no sparger plates, nozzles, orifice plates, baffles, or protrusions so that said large bubbles rather than small bubbles form in the head piece and move from the head piece into the tube.

6. The photobioreactor system as in claim 1, wherein the tail piece is a rigid pipe closed at a distal end and open at a proximal end for fluid communication with the tube, the tail piece having a port in an upper surface of the tail piece and a connector to a vent line for off-gassing from the bioreactor at said tail end.

7. The photobioreactor system as in claim 1 comprising a water line at or near an outer surface of each elongated bioreactor, the water line at or near an outer surface of each elongated bioreactor, the water line extending the length of each elongated bioreactor and being connected to spraying nozzles that spray water on the bioreactor to evaporatively cool the bioreactor.

8. The photobioreactor system as in claim 7, wherein the tube resides in an elongated trough that collects run-off from the water sprayed on the bioreactor.

9. The photobioreactor system as in claim 8, comprising a drain trough under at least a portion of the head piece and adapted to catch liquid flowing from said elongated trough, the drain trough piped to a sewer or other waste treatment.

10. The photobioreactor system as in claim 1 comprising a shade adapted to extend various amounts over each bioreactor to shade the bioreactor from sunshine.

11. The photobioreactor system as in claim 7 comprising a shade adapted to extend various amounts over each bioreactor to shade the bioreactor from sunshine.

12. The photobioreactor system as in claim 1, wherein the tube is divided into multiple tube portions connected together by multiple hollow collars, each collar comprising a collar wall surrounding and defining a hollow interior, two open ends in communication with the hollow interior, and having at least one port through the collar wall into the hollow interior of the collar, so that interior spaces of the multiple tube portions are in fluid communication with the open ends and the hollow interior, and the at least one port is adapted for insertion of a sensing probe through the port and into the hollow interior for monitoring operating conditions in the collar.

13. The photobioreactor system as in claim 12, wherein the sensing probe monitors operating conditions in the collar selected from a group consisting of pH, conductivity, temperature, oxygen content.

14. The photobioreactor of claim 12, wherein the color is rigid.

15. The photobioreactor system as in claim 1, wherein the tube is divided into multiple tube portions connected together by multiple hollow collars, each collar comprising a collar wall surrounding and defining a hollow interior, two open ends in communication with the hollow interior, and having at least one port through the collar wall into the hollow interior of the collar, so that interior spaces of the multiple tube portions are in fluid communication with the open ends and the hollow interior, and the at least one port is adapted for insertion of a sampling syringe through the port and into the hollow interior for sampling the cell suspension in the collar.

16. The photobioreactor of claim 15, wherein the collar is rigid.

17. The photobioreactor system as in claim 1, wherein tube is transparent.

18. The photobioreactor system as in claim 12, wherein tube is transparent and the collars are opaque.

19. The photobioreactor system as in claim 15, wherein tube is transparent and the collars are opaque.

20. The photobioreactor system as in claim 1, further comprising at least one hollow collar around an outside surface of the flexible tube, each collar comprising a collar wall surrounding and defining a hollow interior and having a least one port through the collar wall to the hollow interior of the collar, so that the at least one port is adapted for insertion of a sensing probe or sampling syringe through the port and into the cell suspension in the tube that is received in the collar.

21. The photobioreactor system of claim 1, wherein one or more of the elongated photobioreactor comprises one or more constrictions along the length of the elongated photobioreactor.

22. The photobioreactor system of claim 1, wherein the photobioreactor system further comprises baffles along the length of the elongated photobioreactor.

23. The photobioreactor system of claim 1, wherein the photobioreactor system further comprises one or more block or protrusion members pushing against an outside surface of the flexible tube of the elongated photobioreactor to force the flexible tube into a non-cylindrical shape for preventing or reduced cell settling.

24. The photobioreactor system of claim 1, wherein the cell is algae.

25. A method comprising operating a photobioreactor of claim 1 to culture a cell.

26. The method of claim 25, wherein the cell is algae.

27. A cell produced by the method of claim 25.

28. An algae cell produced by the method of claim 25.
